(19) United States
(12) Patent Application Publication
(10) Pub. No.: US 2015/0246988 A1
(43) Pub. Date: Sep. 3, 2015

(54) METHOD FOR PRODUCING ZWITTERIONIC MONOMERS AND USE OF SAID MONOMERS

(71) Applicant: KARLSRUHER INSTITUT FÜR TECHNOLOGIE, Karlsruhe (DE)

(72) Inventors: Domenic Kratzer, Karlsruhe (DE); Christian Friedmann, Karlsruhe (DE); Joerg Lahann, Ann Arbor, MI (US)

(21) Appl. No.: 14/433,155

(22) PCT Filed: Oct. 23, 2013
(86) PCT No.: PCT/EP2013/072159

§ 371 Date: Apr. 2, 2015

(30) Foreign Application Priority Data
Oct. 24, 2012 (DE) 10 2012 110 156.6

(51) Int. Cl. C08F 120/38; C07C 309/14

(52) U.S. Cl. CPC C08F 120/38; C07C 309/14

(57) ABSTRACT

The present invention relates to a method for producing zwitterionic monomers having variable charge distances m between an anionic group and a cationic group, wherein the dihalogen compounds are reacted with N,N-alkylated amino alcohols to form zwitterionic, monohalogenated monoalcohols, which are then sulfonated by adding metal sulfite salts. The final step of the synthesis is an acid-catalyzed esterification of said zwitterionic monoalcohols with methacrylic acid or acrylic acid to form the zwitterionic monomer.

The present invention relates to a method for producing zwitterionic monomers and use thereof.

Zwitterionic polymer coatings are suitable to generate materials with specific surface properties. Such modified surfaces have been successfully tested, inter alia, for antifouling or antibacterial properties and also as synthetic cell culture matrices for embryonic stem cells. (Poly)sulfobetaine methacrylate-coated materials especially have been found to be particularly suitable. Such (meth)acrylates are compatible with numerous known polymerization methods, inter alia, surface-initiated controlled/living radical polymerizations which are very particularly suitable for modifying surface properties. However, the production of the parent monomeric zwitterionic (meth)acrylates having an anionic sulfonate group is difficult. To date the synthesis of said monomers is limited only to a ring-opening reaction of cyclic sultones.

Owing to the structure of the sultones, the synthesis of the monomers is focused on zwitterionic sulfonates having three or four methylene groups between the positive and negative charge. A monomer having two methylene groups between the charges is only accessible in extremely poor yields. The structure of the monomers, particularly the alkyl chain length between the charges, influences considerably the polymer geometry of the resulting polymer chains and thus has a major influence on their properties. Up to the present time, no syntheses have been described which afford access to the (meth)acrylic-based sulfonates having variable charge distance—i.e. less than three or more than four methylene groups between the positive and negative charge. In the synthesis of the appropriate novel monomers, especially the insertion of the reactive (meth)acrylic group, and the strong polarity and the resulting associated poor solubility of some reactants or products, has proven to be particularly problematic. Monomeric (meth)acrylic sulfonates having more than four methylene groups between the positive and negative charge are therefore unknown.

Owing to the problems mentioned in the synthesis of derivatives of zwitterionic (meth)acrylate monomers, only compounds resulting from ring-openings are known. The most extensive compound library of the corresponding sulfonate substance class is published in P. Koberle, A. Laschewsky, Macromolecules 1994, 27, 2165-2173. This publication is, however, limited to compounds having three or four methylene groups between the positive and negative charge.

A specific synthetic route is known from Y. Terayama et al., Macromolecules 2011, 44, 104-111 which leads to a methacrylate having an alternative internal charge distance. In this case, the N,N-dimethylaminoethyl methacrylate is reacted with vinylsulfonyl chloride to form a monomer having two methylene groups between the positive and negative charge. The disadvantage of this synthesis is the poor yield and the limitation to a charge distance m=2.

Another possibility is presented in J. G. Weers et al., Langmuir 1991, 7, 854-867, by which sulfobetaines can be prepared with variable internal charge distance. However, the synthesis does not take into consideration the insertion of a (meth)acrylic group, but is limited to non-functionalized sulfobetaines.

Further, preparation of zwitterionic sulfonates with three methylene groups between the positive and negative charge is described in JP 10-087 601A.

Moreover, a series of publications on the biological applications of zwitterionic polymers has been reported. A selection showing an overview is given below:

US 2010/0068810 A1, US 2008/0181861 A1, L. G. Villa-Diaz, H. Nandivada, J. Ding, N. C. Nogueira-de-Souza, P. H. Krebsbach, K. S. O'Shea, J. Lahann, G. D. Smith, Nat. Biotechnol. 2010, 28, 581-583, Z. Zhang, S. Chen, Y. Chang, S. Jiang, J. Phys. Chem. B. 2006, 110, 10799-10804, W. K. Cho, B. Kong, I. S. Choi, Langmuir, 2007, 23, 5678-5682, H. Kitano, H. Suzuki, K. Matsuura, K. Ohno, Langmuir 2010, 26, 6767-6774, W. Feng, S. Zhu, K. Ishihara, J. L. Brash, Langmuir 2005, 21, 5980-2987, Z. Zhang, S. Chen, Y. Chang, S. Jiang, Biomacromolecules 2006, 7, 3311-3315.

Additionally, polymerized zwitterionic monomers having a propyl group between the positive and negative charge are used in cell culture according to US 2010/0068810 A1.

Zwitterionic (meth)acrylate monomers having a distance of C1-C6 between the positive and negative charge are used, in accordance with U.S. Pat. No. 8,183,181 B1 after an inverse emulsion polymerization, in boreholes for oil production.

The object of the present invention is to provide a novel, efficient synthetic route for producing zwitterionic (meth)acrylates having variable charge distance or polymers and copolymers resulting therefrom.

This object is achieved by a method for producing zwitterionic monomers having variable charge distances, preferably where m=2-100, particularly preferably m=5-50, especially preferably m=5-20, most preferably where m=5-16 between the anionic and cationic group, wherein zwitterionic sulfonated N,N-alkylated amino alcohols are esterified with acrylic acid or methacrylic acid in acrylic acid or methacrylic acid in the presence of a further acid.

Index m defines the number of carbon atoms between the two halogen atoms.

To produce the zwitterionic N,N-alkylated amino alcohols, halogen compounds are reacted with N,N-alkylated amino alcohols to give halogenated N,N-alkylated amino alcohols.

Dihalogen compounds are preferably used, particularly dihaloalkanes. To produce the zwitterionic sulfonated N,N-alkylated amino alcohols, the zwitterionic N,N-alkylated amino alcohols are sulfonated by adding metal sulfites.

In one embodiment, halosulfonates are reacted with N,N-alkylated amino alcohols to produce the zwitterionic sulfonated N,N-alkylated amino alcohols.

The final step of the synthesis is an acid-catalyzed esterification of said zwitterionic sulfonated alcohols, preferably monoalcohols, with methacrylic acid or acrylic acid in acrylic acid or methacrylic acid to form the zwitterionic monomer.

The result is the production of a monomer of the formula I

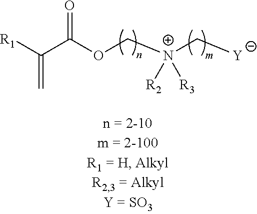

The index n is preferably between 1-10, preferably 2-8, particularly preferably 1-5, particularly 2, 3 or 4. Preference is given to CH₃ (Me), CH₃CH₂ (Et) as alkyls R₁. However, the reaction would also work with longer chain acrylic acids.

The charge distance m is 2-100, 7-100, 8-100, 10-100, 12-100, 10-90, 12-80, particularly preferably m=5-50, 7-50, 8-50, 10-50, 12-50, especially preferably m=5-20, 7-20, 8-20, 10-20, 12-20, most preferably m=5-16, 7-16, 8-16, 10-16, 12-16.

The residues R_{2, 3} may be methyl, ethyl, propyl, isopropyl, butyl or pentyl. All alkyl residues may also be mixed, e.g.: 1×methyl and 1×ethyl on the nitrogen or 1×ethyl and 1×butyl.

All inorganic sulfites can be used in accordance with the invention. Suitable metal sulfites are preferably alkali metal sulfites, particularly potassium sulfite and sodium sulfite. Particular preference is given to sodium sulfite. In one alternative, calcium sulfite, magnesium sulfite, barium sulfite, beryllium sulfite and transition metal sulfites are suitable.

The zwitterionic sulfonated alcohol obtained by the method described is, in a further aspect of the invention, esterified in acrylic acid under acid catalysis. The esterification may also be carried out using methyl or ethylacrylic acids. The two method steps described can be carried out such that a zwitterionic monomer suitable for further polymerization is formed by esterification. As a result of the method, a zwitterionic sulfonate with terminal alcohol functionality is attained in the second stage and the final esterification of the alcohol with acrylic acid in the third stage achieves the finished monomer:

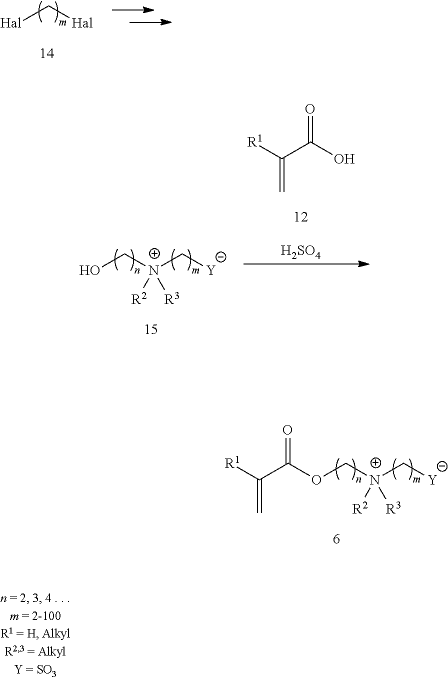

The residues mentioned above are used for alkyls R₁. The abovementioned residues are likewise suitable as alkyl residues R_{2, 3}.

The charge distance is 2-100, preferably 5-50, particularly particularly preferably 5-20, especially preferably 5-16.

The halogen compounds used are preferably haloalkanes, preferably dihaloalkanes.

The halogen components HAL used are preferably bromine, chlorine, iodine or mixtures of one or more of these substances. For example, dibromoalkanes, dichloroalkanes, diiodoalkanes with m=5-20, also corresponding mixed haloalkanes, i.e. chlorobromoalkanes or bromoiodoalkanes, cyclic dihalogens such as 1,4-dibromocyclohexane and corresponding mixed halogenized cyclic halogens and also aromatic dihalogens can be used. Instead of halogens, ditosylates or ditriflates or combinations thereof can also be used.

Likewise, haloalkyl tosylates or triflates or combinations thereof can be used.

In the following example, the dihaloalkane used is a bromine-containing compound:

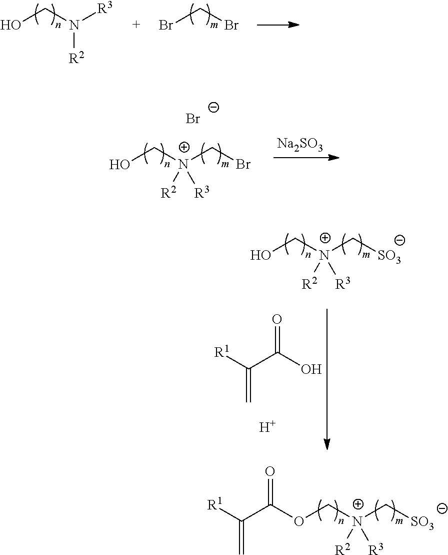

As a result, zwitterionic (meth)acrylates are preferably produced.

Preferred catalytic acids include all sulfuric acid derivatives. Likewise, organic sulfonic acids can be used. That is to say, in addition to sulfuric acid, para-toluenesulfonic acids are also useful. Further examples are: methanesulfonic acid, benzenesulfonic acid, camphorsulfonic acid and/or 2-(cyclohexylamino)ethanesulfonic acid.

In the acid-catalyzed esterification, carboxylic acids function both as solvent and as reagent. For example, zwitterionic (meth)acrylic-based monomers can be produced by the method according to the invention according to the following scheme.

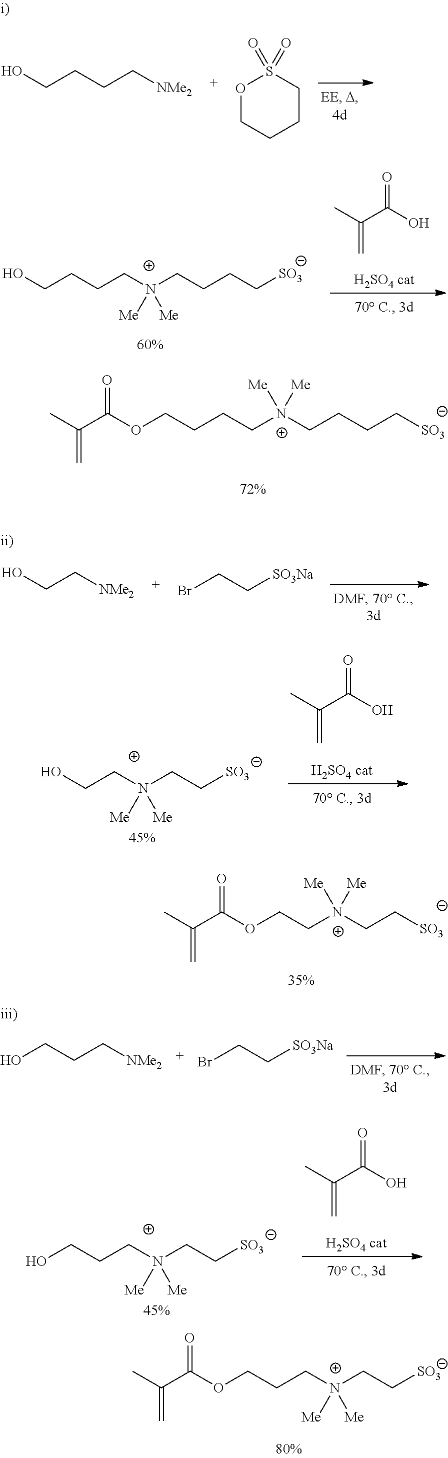

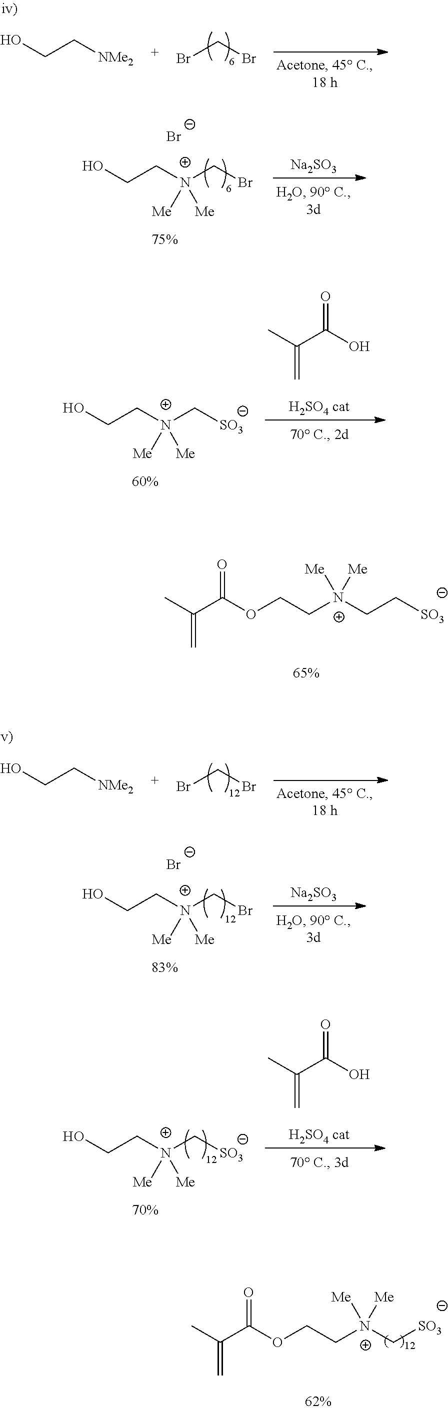

Further attainable compounds which can be obtained by the method according to the invention are listed below.

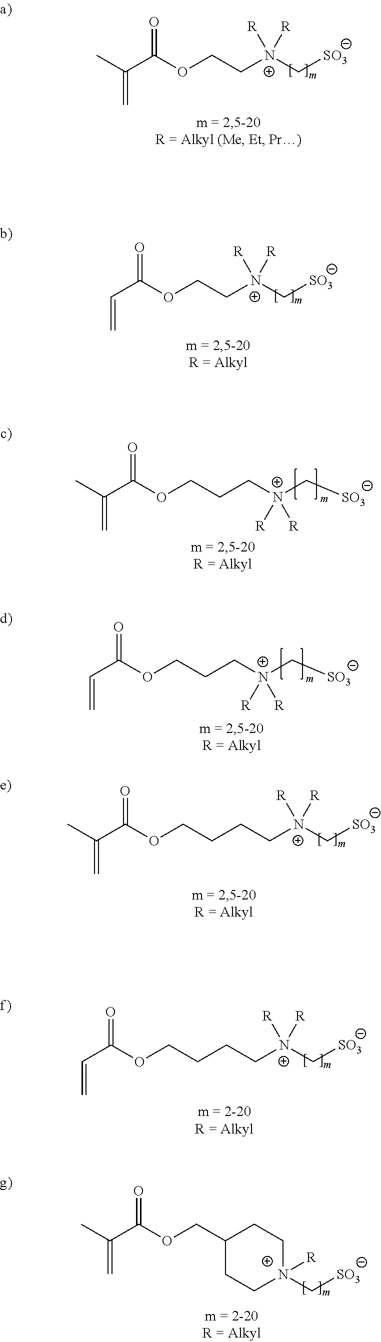

The abovementioned residues are also useful as alkanes

In a further aspect of the invention, the methacrylic-based monomers produced by the method according to the invention are claimed. In this case, this takes the form of monomers of the general formula II

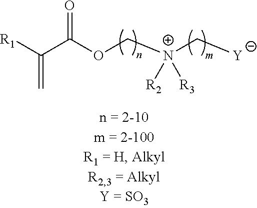

The index n is preferably between 1-10, preferably 2-8, particularly preferably 1-5, particularly 2, 3 or 4. Preference is given to CH₃ (Me), CH₃CH₂ (Et) as alkyls R₁.

The charge distance according to the invention is—

m=7-100, 8-100, 10-100, 12-100, 10-90, 12-80, particularly preferably m=5-50, 7-50, 8-50, 10-50. 12-50 especially preferably m=5-20, 7-20, 8-20, 10-20, 12-20 most preferably m=5-16, 7-16, 8-16, 10-16, 12-16.

In a further alternative of the invention, zwitterionic alcohols of the formula:

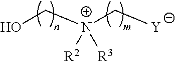

are also claimed. The residues are as defined above. The same applies to n and m.

Furthermore, the invention in one alternative comprises two methacrylates of the formula

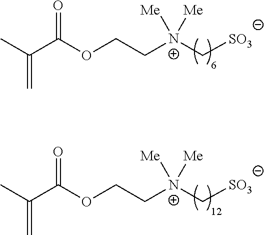

It is possible to achieve the production of (meth)acrylic-functionalized zwitterions with little effort in accordance with the invention. Numerous starting compounds and numerous variations of dihalogen compounds can be used. Important in intermediate stages in accordance with the invention are the ammonium alkyl sulfonates having alcohol functionality Polymerizable products can then be obtained by the introduction of the (meth)acrylic groups.

Polymers and copolymers can be produced from the monomers described. These are suitable for developing novel biomaterials. Novel biomaterials with specific properties are provided in accordance with the invention which are suitable, inter alia, as a synthetic matrix for stem cell cultures. The zwitterionic materials according to the invention result in biomaterials with completely new properties. Biomaterials are characterized by their biocompatibility. The zwitterionic monomers according to the invention and monomers produced by the method according to the invention are preferably used for producing biomaterials with improved biocompatibility.

### EXAMPLES

### 12-Bromo-N-(2-hydroxyethyl)-N,N-dimethyldodecane-1-ammonium bromide

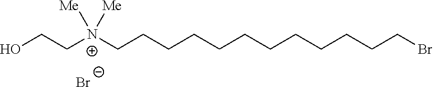 [SMILES: C[N+](C)(CCO)CCCCCCCCCCCCBr ; [Br-]]

1,12-Dibromododecane (30.0 mmol, 9.84 g, 4.00 equiv.) were dissolved in 40 mm of acetone and heated to 45° C. 2-(dimethylamino)ethanol (7.50 mml, 0.75 ml, 1.00 equiv.) were added slowly with stirring to this mixture over a period of 6 hours. The reaction mixture was stirred for a further 18 hours at 45° C. After cooling to room temperature, the dark brown oil was separated by decanting and subsequent filtration of the solvent. Acetone was separated from the liquid phase by evaporation. The oil residue was dissolved in 100 ml of ethyl acetate. The product was extracted from this mixture with water (3×50 ml). The aqueous phases were combined and dried by evaporation such that the product was obtained as a brown, waxy substance.

Yield 2.60 g (83%).

—R_{f}=0.08 (methanol).—¹H-NMR (500 MHz, MeOD): 4.00-3.97 (m, 2H, OCH₂), 3.49-3.38 (m, 6H, 2×NCH₂, BrCH₂), 3.16 (s, 6H, 2×NCH₃), 1.87-1.76 (m, 4H, 2×CH₂), 1.46-1.33 (m, 16H, 8×CH₂) ppm.—¹³C-NMR (125 MHz, MeOD): δ6.87 (−, CH₂), 66.55 (−, CH₂), 56.92 (−, CH₂), 52.22 (+, 2×NCH₃), 34.50 (−, CH₂), 34.04 (−, CH₂), 30.63 (−, CH₂), 30.61 (−, 2×CH₂), 30.56 (−, CH₂), 30.25 (−, CH₂), 29.88 (−, CH₂), 29.20 (−, CH₂), 27.45 (−, CH₂), 23.69 (−, CH₂) ppm.

### 12-[(2-Hydroxyethyl)dimethylammonio]dodecane sulfonate

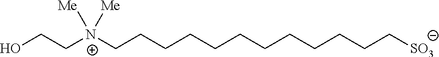

### 12-Bromo-N-(2-hydroxyethyl)-N,N-dimethyldodecane-1-ammonium bromide (4)

(4.94 mmol, 2.06 g, 1.00 equiv.) were dissolved in 15 ml of water and heated under reflux. After 10 minutes, sodium sulfite (5.93 mmol, 747 mg, 1.20 equiv.) was added to this solution. The reaction mixture was then stirred for a further 72 hours at 90° C. After cooling to room temperature, the solvent was removed under reduced pressure. The white residue was dissolved in 100 ml of methanol and the mixture was filtered. The methanol was then removed under reduced pressure and the crude product absorbed on silica and subjected to flash chromatography (silica, methanol). The product was thus obtained as a white solid.

Yield: 1.17 g (70%).

—R_{f}=0.23 (methanol), 0.14 (dichloromethane/methanol 2/1).—¹H-NMR (500 MHz, MeOD): 4.00-3.97 (m, 2H, OCH₂), 3.47-3.45 (m, 2H, NCH₂), 3.41-3.38 (m, 2H, NCH₂), 3.15 (s, 6H, 2×NCH₃), 2.79-2.76 (m, 2H, CH₂SO₃), 1.83-1.75 (m, 4H, 2×CH₂), 1.44-1.33 (m, 16H, 8×CH₂) ppm. —¹³C-NMR (125 MHz, MeOD: 66.87 (−, CH₂), 66.52 (−, CH₂), 56.92 (−, CH₂), 52.73 (−, CH₂), 52.19 (+, 2×NCH₃), 30.23 (−, 2×CH₂), 30.20 (−, CH₂), 30.08 (−, 2×CH₂), 30.00 (−, CH₂), 29.58 (−, CH₂), 27.34 (−, CH₂), 23.59 (−, CH₂) ppm.—FT-IR (ATR): v=3420 (w), 3298 (w), 2961 (vw), 2914 (w), 2846 (w), 1638 (vw), 1482 (w), 1463 (w), 1354 (vw), 1215 (w), 1171 (m), 1096 (m), 1072 (w), 1039 (m), 1004 (w), 986 (w), 970 (w), 924 (w), 791 (w), 601 (m), 540 (w), 521 (m), 450 (w) cm⁻¹.—MS (FAB), m/z (%): 338.2 (100) [M]⁺, 256.4 (11), 154.3 (9), 89.4 (10).—HR-MS (FAB) calcd for C₁₆H₃₆NO₄S: 338.2365. found 338.2368 [M]⁺.

### 12-[[2-(Methacryloyloxy)ethyl](dimethyl)ammonio]-1-dodecane sulfonate (I and II)

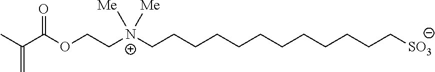

An oven-dried, 25 ml two-necked flask fitted with a condenser was evacuated and filled with argon and then charged with 6 ml of methacrylic acid. After heating to 70° C.,

12-[(2-hydroxyethyl)dimethylammonio]dodecane sulfonate (5) (0.90 mmol, 304 mg) and hydroquinone (20 mg) were added with stirring to the flask. After 30 minutes, 5 drops of sulfuric acid were added to this suspension using a 1 ml syringe. The reaction mixture was stirred for 72 hours at 60° C. After cooling to room temperature, the liquid phase was separated from the brown oil by decanting. The oily residue was then dried under vacuum, while the liquid phase was evaporated to dryness. After one hour, both residues were dissolved in methanol (50 ml) which were combined and absorbed onto silica. The product was then obtained from flash chromatography (silica, dichloromethane/methanol 2/3) as a further solid.

Yield: 226 g (62%).—R_{f}=0.35 (methanol).—¹H-NMR (500 MHz, MeOD): 6.16-6.15 (m, 1H, C═CH₂), 5.74-5.72 (m, 1H, C═CH₂), 4.62-4.60 (m, 2H, OCH₂), 3.76-3.74 (m, 2H, NCH₂), 3.43-3.39 (m, 2H, NCH₂), 3.17 (s, 6H, 2×NCH₃), 2.79-2.76 (m, 2H, CH₂SO₃), 1.97 (s, 3H, H₂C═CCH₃), 1.83-1.76 (m, 4H, 2×CH₂), 1.44-1.33 (m, 16H, 2×CH₂) ppm.—¹³C-NMR (125 MHz, MeOD): 167.69 (C_{quart}, C═O), 137.15 (C_{quart}, C═CH₂), 127.26 (−, C═CH₂), 66.66 (−, CH₂), 63.79 (−, CH₂), 59.12 (−, CH₂), 52.74 (−, CH₂), 51.92 (+, 2×NCH₃), 30.26 (−, 2×CH₂), 30.11 (−, 2×CH₂), 30.08 (−, CH₂), 29.61 (−, 2×CH₂), 27.36 (−, CH₂), 25.90 (−, CH₂), 23.61 (−, CH₂), 18.43 (+, H₂C═CCH₃) ppm.—MS (FAB), m/z (%): 406.1 (100) [M]⁺, 324.2 (8), 154.3 (9), 113.4 (34).—HR-MS (FAB) calcd for C₂₀H₄₀NO₅S: 406.2627. found 406.2629 [M]⁺.

1-15. (canceled)

16. A method for producing a zwitterionic monomer having a variable charge distance m between an anionic group and a cationic group, wherein the method comprises esterifying a zwitterionic sulfonated N,N-alkylated amino alcohol with acrylic acid or methacrylic acid in acrylic acid or methacrylic acid in the presence of a further acid.

17. The method of claim 16, wherein a halogen compound is reacted with an N,N-alkylated amino alcohol to afford a zwitterionic halogenated N,N-alkylated amino alcohol.

18. The method of claim 17, wherein a dihalogen compound is employed as the halogen compound.

19. The method of claim 17, wherein a halohydrocarbon is employed as the halogen compound.

20. The method of claim 19, wherein a dihaloalkane is employed.

21. The method of claim 16, wherein a at least one of a ditosylate and a ditriflate is reacted with an N,N-alkylated amino alcohol.

22. The method of claim 16, wherein a zwitterionic N,N-alkylated amino alcohol is sulfonated by addition of a metal sulfite to produce a zwitterionic sulfonated N,N-alkylated amino alcohol.

23. The method of claim 22, wherein the metal sulfite is selected from one or more of alkali metal sulfites, alkaline earth metal sulfites, or transition metal sulfites.

24. The method of claim 16, wherein a halosulfonate is reacted with an N,N-alkylated amino alcohol to afford a zwitterionic sulfonated N,N-alkylated amino alcohol.

25. The method of claim 16, wherein the further acid is selected from sulfuric acid, para-toluenesulphonic acid, derivatives thereof.

26. A zwitterionic monomer of formula:

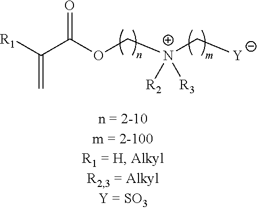

27. The zwitterionic monomer of claim 26, wherein R_{2,3} represents one or more of methyl, ethyl, propyl, isopropyl, pentyl.

28. The zwitterionic monomer of claim 26, wherein m=7-50.

29. The zwitterionic monomer of claim 26, wherein R₁ represents H, methyl or ethyl.

30. The zwitterionic monomer of claim 26, wherein n is 2, 3 or 4.

31. The zwitterionic monomer of claim 27, wherein m=7-50, R₁ represents H, methyl or ethyl and n is 2, 3 or 4.

32. A method for producing copolymers or polymers or synthetic matrices for cell cultures, wherein the method comprises employing the zwitterionic monomer of claim 26 as a monomer for producing the copolymers or polymers.

33. A method for producing synthetic matrices for stem cell cultures, wherein the method comprises employing the zwitterionic monomer of claim 26 as a starting material for the synthetic matrices.
